(19) United States
(12) Patent Application Publication
(10) Pub. No.: US 2011/0028845 A1
(43) Pub. Date: Feb. 3, 2011

(54) Reconfigurable Ultrasound Array with Low Noise CW Processing

(76) Inventors: Bruno Hans Haider, Niskayuna, NY (US); Kjell Kristoffersen, Horten (NO); Robert Gideon Wodnicki, Niskayuna, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(21) Appl. No.: 12/534,099

(22) Filed: Jul. 31, 2009

(51) Int. Cl. A61B 8/14

(52) U.S. Cl. 600/459

(57) ABSTRACT

An ultrasound transducer probe is disclosed comprising: an array of ultrasound transducer elements, each ultrasound transducer element associated with a corresponding unit transducer cell for providing transmit and receive functions, each unit transducer cell including a cell transmit/receive switch connected to a low voltage switch matrix via a low voltage transmit path; and a plurality of microelectronic cross-point switches for switching an externally generated analog transmit signal to one or more of the low voltage transmit paths.

### BACKGROUND OF THE INVENTION

The subject matter disclosed herein generally relates to reconfigurable sensor arrays and, in particular, to a reconfigurable ultrasound transducer array.

The present state of the art discloses a number of imaging systems having ultrasound sensor arrays configured to produce two-dimensional (2-D) images of a human heart in real-time inside a subject's body. Typical applications for such imaging systems include diagnosis and the monitoring of interventional procedures in, for example, Trans-Esophoegeal Echocardiography, Intra-Cardiac Echocardiography, and Intra-Vascular Ultrasound.

Imaging systems that are capable of producing three-dimensional (3-D) images in real-time typically utilize beam-forming electronics that occupy a larger volume than a corresponding 2-D imaging system and are thus not practical for placement inside the body to image the human heart. Some conventional ultrasound probes utilize micro-motors to actuate 2-D transducers placed inside the body to acquire 3-D imaging volumes in real time. However these micro-motor probes do not have the ultrasound beam agility of an electronically steered ultrasound probe and have not demonstrated the reliability offered by completely solid-state probes.

A reconfigurable sensor array offers a method to reduce the requirements on size and power for the beam-forming electronics, but uses high voltage switches at the transducer. Accordingly, such arrays suffer from the large size of such devices and are therefore limited to smaller sizes or higher on-resistance which leads to unwanted signal attenuation and delay. It is also possible to use a pulser switch matrix in which each transducer element is driven directly by a local pulser circuit while the transmit timing signal is distributed throughout the array using a low voltage switching network. While this solution can work well for B-mode imaging, it may not have adequate noise performance for high quality Doppler imaging.

The inventors herein have recognized a need for a high quality Doppler-capable reconfigurable ultrasound array for use in imaging the heart from inside the body.

### BRIEF DESCRIPTION OF THE INVENTION

An ultrasound transducer probe comprises: an array of ultrasound transducer elements, each ultrasound transducer element associated with a corresponding unit transducer cell for providing transmit and receive functions, each unit transducer cell including a cell transmit/receive switch connected to a low voltage switch matrix via a low voltage transmit path; and a plurality of microelectronic cross-point switches for switching an externally generated transmit control signal to one or more of the low voltage transmit paths.

An ultrasound transducer probe system comprising: a probe including, an array of ultrasound transducer elements; a plurality of unit transducer cells, each ultrasound transducer element coupled to a corresponding unit transducer cell; a transmit channel line for conducting transmit control signals from an ultrasound driver to a low voltage switch matrix in each of the unit transducer cells, the low voltage switch matrix for switchably providing the transmit control signal to a corresponding ultrasound transducer element via a low-voltage electrical path; and a programming circuit connected to each low voltage switch matrix by a system channel line.

A method for monitoring an interventional procedure inside a patient, the method comprising: providing an ultrasound transducer probe having a reconfigurable ultrasound transducer array; guiding the probe to a region of interest inside the patient; providing a transmit control signal to a transducer array through a switch matrix via a low-voltage electrical path; providing a control signal to the reconfigurable ultrasound transducer array for electronically steering an ultrasound beam produced by the ultrasound transducer probe; and imaging the interior of the patient via the ultrasound beam to obtain a three-dimensional, real-time image of the region of interest.

Other systems and/or methods according to the embodiments will become or are apparent to one with skill in the art upon review of the following drawings and detailed description. It is intended that all such additional systems and methods be within the scope of the present invention, and be protected by the accompanying claims.

### BRIEF DESCRIPTION OF THE DRAWINGS

FIG. 1 shows a probe system with a reconfigurable ultrasound transducer array in communication with a programming circuit and a transmit/receive system, in accordance with an exemplary embodiment of the present invention;

FIG. 2 shows a two-dimensional ultrasound transducer element array used in the probe system of FIG. 1;

FIG. 3 is an exploded isometric diagrammatical illustration of a portion of the two-dimensional ultrasound transducer element array of FIG. 2;

FIG. 4 is a diagrammatical illustration of an exemplary embodiment of a unit transducer cell in the probe system of FIG. 1 configured for ultrasound transmission using local high-voltage pulse timing in the reconfigurable ultrasound transducer array of FIG. 2;

FIG. 5 is a flow diagram illustrating operation of the probe system of FIG. 4;

FIG. 6 is a diagrammatical illustration of an exemplary embodiment of a unit transducer cell in the probe system of FIG. 1 configured for ultrasound transmission using external pulse timing in the reconfigurable ultrasound transducer array of FIG. 2;

FIG. 7 shows a level shifter configured for use in the unit transducer cell of FIG. 6; and

FIG. 8 is a flow diagram illustrating operation of the probe system of FIG. 6.

### DETAILED DESCRIPTION OF THE INVENTION

This invention describes a reconfigurable switch matrix based ultrasound probe for producing high quality Doppler ultrasound images of the heart using a relatively compact and low-power ultrasound beamforming system. The system is especially useful for imaging the heart from inside the body. This method of imaging may provide an increased available image volume and an improved signal-to-noise ratio for use in catheter-based or endoscopy-based echo-cardiography imaging. The disclosed probe configuration may provide several advantages including, for example: 1) improved reliability as moving parts are not used; 2) lower power requirements; 3) smaller physical size; 4) greater ultrasound beam agility; 5) lower-cost solid-state implementation; and 6) higher quality Doppler imaging capabilities.

In particular, the reconfigurable ultrasound probe disclosed herein provides a reconfigurable array that can be adapted to, for example, an intercardiac echocardiography (ICE) probe, an intravascular ultrasound (IVUS) probe, or a trans-esophoegeal echocardiography (TEE) probe. The reconfigurable array utilizes timing signals both to control local high voltage pulsers and to drive the transducers themselves in a low voltage Continuous Wave (hereinafter CW) mode. A level-shifter circuit may be included to allow signals having voltage levels greater than logic levels to be passed through a low-voltage reconfigurable array switch network.

Standard B-mode imaging is accomplished by configuring the switch matrix for a given aperture to realize rings (or arcs for steering) which cause the beam to be focused in front of the array. An acoustic beam is transmitted by the array in response to a low voltage timing signal which is propagated throughout the switch matrix. Each of the rings corresponds to a unique ultrasound transmit channel where all elements connected to one ring will transmit sound at the same phase. Within each cell there is a discriminator which decodes the low voltage signal and generates drive signals to control a high voltage pulser which then drives the ultrasound transducer.

The invention also provides a switch gate drive level-shifter circuit which enables the ultrasound beamforming system to transmit continuous wave ultrasound pulses through the low voltage switch network which are larger than what might ordinarily be tolerated according to the required logic levels for the device. In this way, high quality, very low-noise transmit timing signals can be used to control blood flow imaging by circumventing local pulser and timing circuitry.

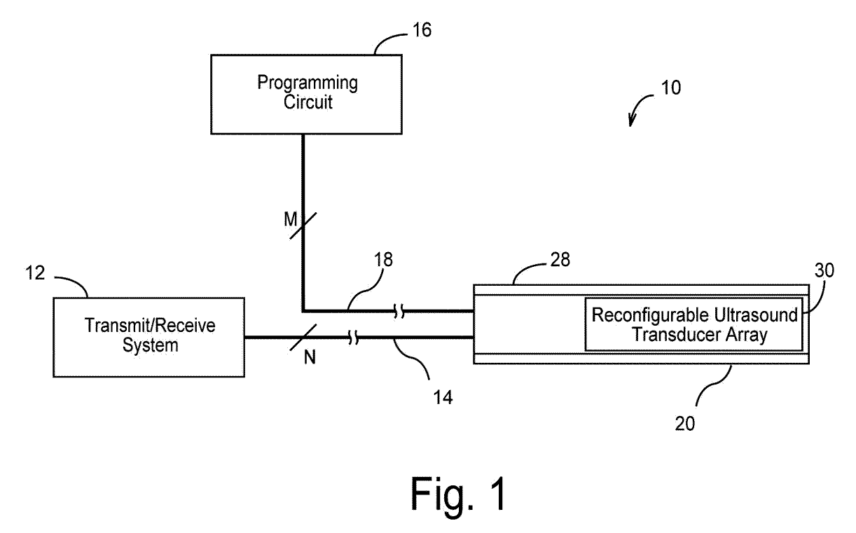

FIG. 1 shows a probe system 10, in one aspect of the present invention. The probe system 10 includes an ultrasound transducer probe 20 in electronic communication with (i) a transmit/receive system 12 via a plurality of ‘N’ analog paths forming a transmit channel line 14, and (ii) a programming circuit 16 via a plurality of ‘M’ digital paths forming a system channel line 18. The programming circuit 16 functions to program switches in the ultrasound transducer probe 20 to be in either an “ON” state or an “OFF” state, or to effect a “NO_CHANGE” in state. The switch configuration functions to electronically steer the beam produced by a reconfigurable ultrasound transducer array 30, which may be disposed in a catheter sleeve 28. The programming circuit 16 may further be used to configure and control ultrasound beamforming by the reconfigurable ultrasound transducer array 30, and to receive ultrasound imaging information, such as may be provided by ultrasound waves reflected from inside a patient (not shown).

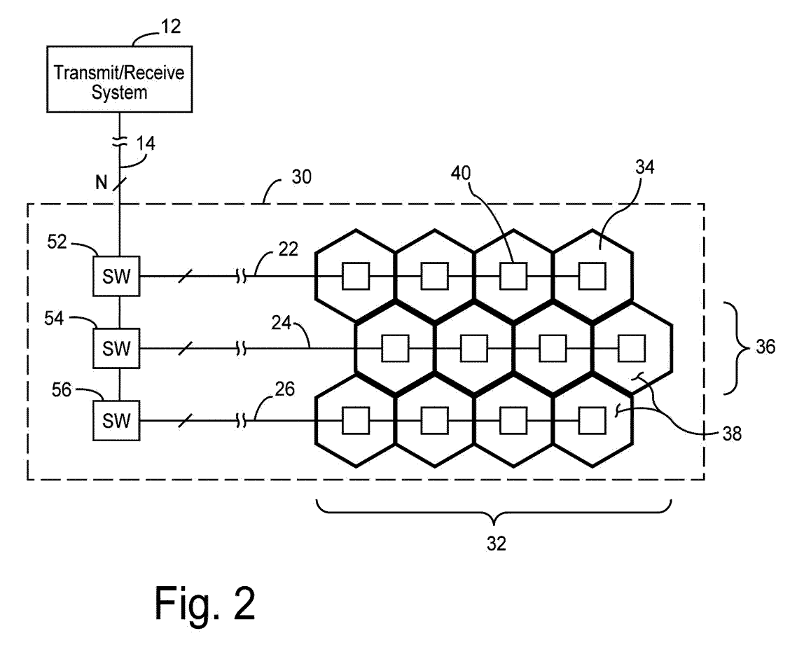

The reconfigurable ultrasound transducer array 30 may comprise a one-dimensional element array (not shown) or a two-dimensional array, as represented by the ultrasound transducer element array 32 of twelve ultrasound transducer elements 34, in FIG. 2, wherein each of the ultrasound transducer elements 34 in the ultrasound transducer element array 32 is associated with and controlled via a corresponding unit transducer cell 40 disposed on the respective ultrasound transducer element 34. The unit transducer cell 40 may also be used for data readout from the ultrasound transducer element 34. The ultrasound transducer element 34 may comprise a piezoelectric transducer (PZT) or a capacitive micro-machined ultrasound transducer (cMUT), for example. The relatively small size of the reconfigurable ultrasound transducer array 30 thus allows for placement of the ultrasound transducer probe 20 within a small cavity, such as inside a human heart chamber, during an imaging procedure.

In an exemplary embodiment, each ultrasound transducer element 34 is substantially hexagonal in shape to provide for a close-packing configuration in the ultrasound transducer element array 32, although the present invention is not limited to this configuration and other one-dimensional or two-dimensional ultrasound transducer element array geometries may be used. An active surface 38 (i.e., the emitting surface of the ultrasound transducer element array 32) may be defined by the aggregate of the individual surfaces of the transducer elements 34 comprising the ultrasound transducer element array 32. The active surface 38 both emits ultrasound beams and receives ultrasound beam echoes, and may be substantially planar, convex, or concave to function with a specified ultrasound waveform configuration.

Alternatively, the active surface 38 may comprise connected planar, convex, or concave surface sections for greater flexibility in fabrication of the ultrasound transducer element array 32. The plurality of ultrasound transducer elements 34 in the ultrasound transducer element array 32 may be interconnected by a series of microelectronic switches in a switch matrix, as explained in greater detail below, and as disclosed in commonly-assigned U.S. Pat. No. 6,865,140 “Mosaic arrays using micro-machined ultrasound transducers,” incorporated in its entirety herein by reference.

In the exemplary embodiment shown, the ultrasound transducer element array 32 comprises twelve ultrasound transducer elements 34 arranged in three rows but it should be understood that any one- or two-dimensional configuration can be used for the ultrasound transducer element array 32, with more or fewer transducer element rows and more or fewer ultrasound transducer elements in each row, depending upon the particular ultrasound application desired. In an alternative exemplary embodiment (not shown), the transducer element array 32 may comprise a 32×32 array of ultrasound transducer elements 34. As appreciated by one skilled in the relevant art, the reconfigurable ultrasound transducer array 30 enables the dynamic connection and re-connection of groups of selected ultrasound transducer elements 34 so as to provide desired acoustic transmission and receiving patterns during operation of the ultrasound transducer probe 20 (shown in FIG. 1).

Control signals may be provided to the reconfigurable ultrasound transducer array 30 via a plurality of microelectronic cross-point switches, here exemplified by cross-point switches 52, 54, and 56. That is, one cross-point switch may be used for each transducer element row 36. Each of the cross-point switches 52, 54, and 56 functions to connect one or more of the analog paths in the transmit channel line 14 to one or more transmit/receive (T/R) lines in respective T/R busses 22, 24, and 26. The T/R bus 24, for example, thus connects the transmit/receive system 12 to a series of unit transducer cells 40 associated with respective ultrasound transducer elements 34 in the common transducer element row 36.

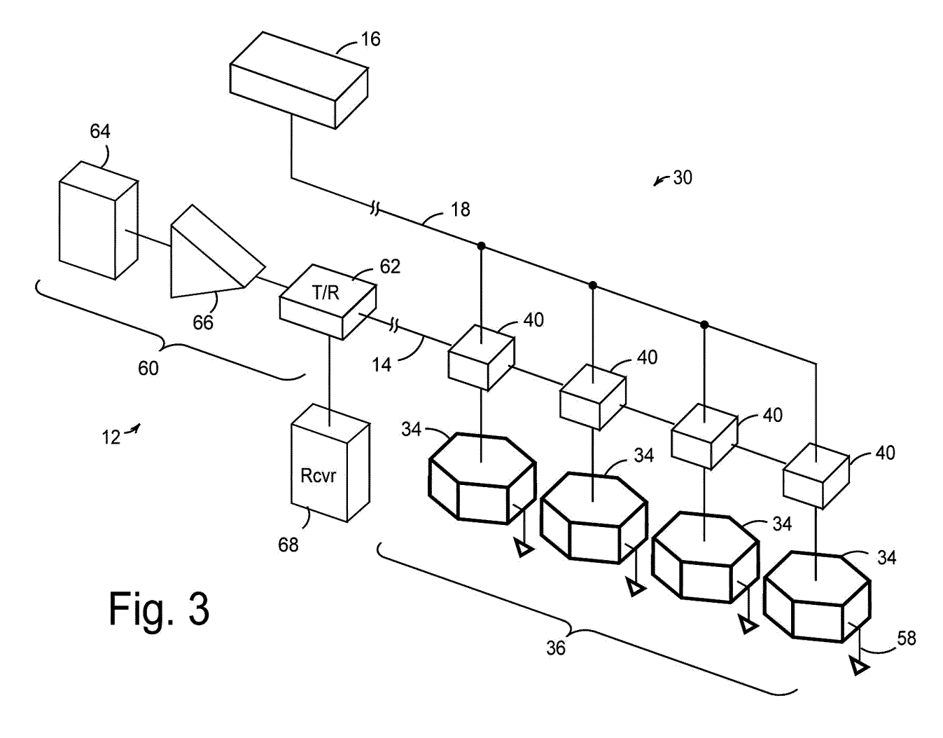

As shown in greater detail in the exploded diagrammatical isometric illustration of FIG. 3, the transmit/receive system 12 provides analog signals to the unit transducer cells 40 via the transmit channel line 14, and the programming circuit 16 provides digital signals via the system channel line 18. The transmit/receive system 12 may comprise a transmit control signal generator 60 and an optional receiver 68. The transmit control signal generator 60 may include a signal generator 64, an ultrasound driver 66, and a transmit/receive switch 62 for selectively providing transmission signals to the reconfigurable ultrasound transducer array 30 via the transmit channel line 14. Note that only one transducer element row 36 of the reconfigurable ultrasound transducer array 30 is shown for clarity of illustration.

The transmit/receive switch 62 can change states to either allow the transmission of signals from the signal generator 64 to selected unit transducer cells 40 and ultrasound transducer elements 34, or to provide signals acquired to the ultrasound receiver 68. Each ultrasound transducer element 34 in the ultrasound transducer element row 36 may also have a local ground 58. It can be appreciated by one skilled in the relevant art that phase noise and timing errors may be produced in the reconfigurable ultrasound transducer array 30 as a result of decoding processes and the use of high-impedance high-voltage transmitters. Such phase noise and propagation errors can be especially detrimental when trying to image blood flow using Doppler processing, for example. Accordingly, it is advantageous that, to reduce noise, the low-voltage signal paths communicating with the system channel line 18 circumvent the high voltage electrical paths providing transmission signals to the ultrasound transducer elements 34, as disclosed below.

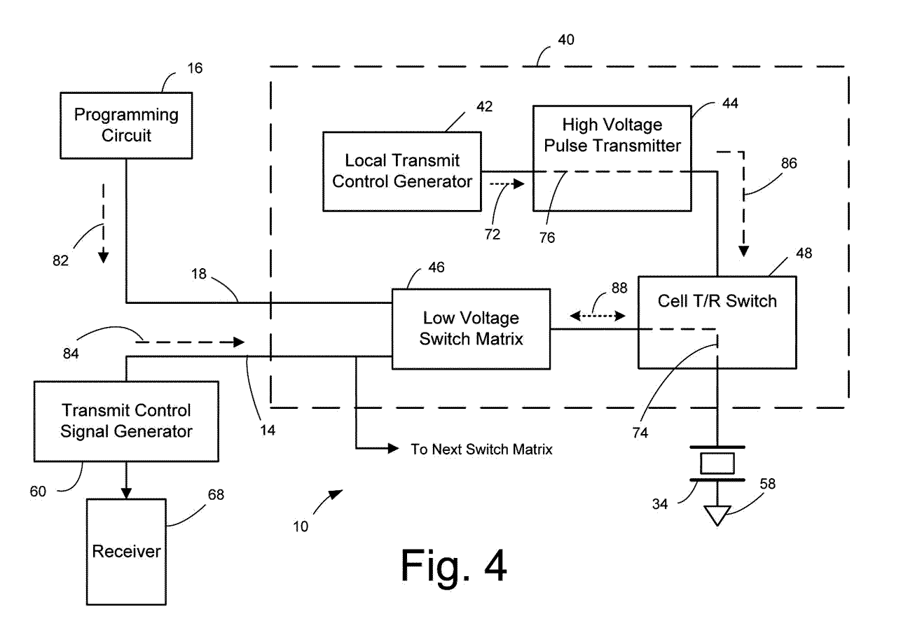

There is shown in FIG. 4 a diagrammatical illustration of an exemplary embodiment of the probe system 10 in which the unit transducer cell 40 is configured for ultrasound transmission using local high-voltage pulse timing. The cross-point switches 52, 54, and 56 are not shown, for clarity of illustration. Digital control signals 82 from the programming circuit 16 may be provided to a low voltage switch matrix 46 on the system channel line 18. Analog transmit signals 84 from the transmit control signal generator 60 may be provided to the low voltage switch matrix 46 on the transmit channel line 14. A local transmit control generator 42 functions to control operation of a high voltage pulse transmitter 44 as a transmission signal 86 is provided to the ultrasound transducer element 34 via a cell transmit/receive (T/R) switch 48.

The low voltage switch matrix 46 may operate in the range of about 2.5 to 5.0 volts using, for example, CMOS devices. The low voltage switch matrix 46 can be switched to pass the analog transmit signal 84 to the cell T/R switch 48 in the unit transducer cell 40 and thereby control operation of the corresponding ultrasound transducer element 34. The electrical path connecting the transmit control signal generator 60 to the low voltage switch matrix 46 and to the cell T/R switch 48 may define a low-voltage transmit path 74. Accordingly, transmit/receive signals 88 may travel between the low voltage switch matrix 46 and the ultrasound transducer element 34 via the low voltage transmit path 74.

The local transmit control generator 42 in the unit transducer cell 40 may provide a pulse transmission signal 72 to control the high voltage pulse transmitter 44, which may operate in a B-mode or in a pulsed wave (PW) Doppler mode, for example. As understood in the relevant art, B-mode imaging includes transmitting a repeated pattern of a relatively small number of pulses, such as one to ten pulses, at a standard rate (i.e., Pulse Repetition Frequency), to acquire data for display as two-dimensional or three dimensional tomographic images. In comparison, PW Doppler operation can be used to obtain velocity data, such as blood flow information. It can be appreciated by one skilled in the art that B-mode imaging may be used to provide probe guidance prior to subsequent probe operation in the PW mode. The transmission signal 86 from the high voltage pulse transmitter 44 may range from about thirty to about five hundred volts. The electrical path from the local transmit control generator 42 to the cell T/R switch 48 may define a high-voltage transmit path 76. The cell T/R switch 48 may also function to isolate the low voltage switch matrix 46 from the high-voltage transmission signals generated by the high voltage pulse transmitter 44. It can be appreciated that high quality, low-noise transmit timing signals on the low voltage transmit path 74 thus circumvent the signals from the local transmit control generator 42 on the high-voltage transmit path 76.

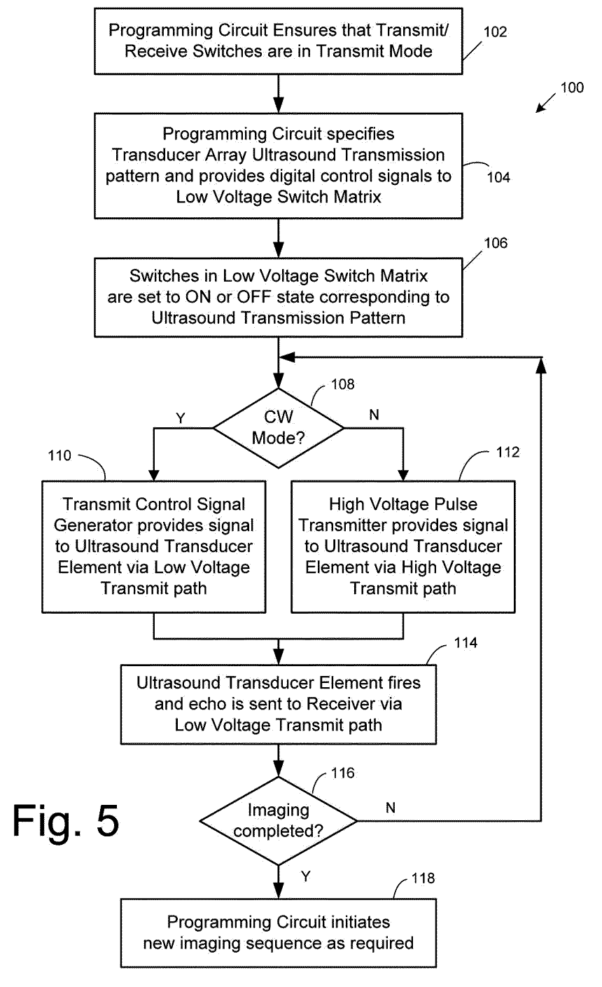

In an exemplary mode of operation, the probe system 10 may function in accordance with a flow diagram 100, shown in FIG. 5. The local transmit control generator 42 verifies or ensures that the cell T/R switch 48 is in a transmit mode, at step 102. The programming circuit 16 may specify an ultrasound transmission pattern for the reconfigurable ultrasound transducer array 30, in step 104. Corresponding digital control signals 82 are provided to the low voltage switch matrices 46 in the unit transducer cells 40 in the ultrasound transducer element array 32. Switches in each low voltage switch matrix 46 are programmed to change state to “ON” or “OFF,” or to remain unchanged in response to a “NO_CHANGE” control signal, at step 106. It should be understood that steps 102, 104, and 106 may be performed in any order, or may be executed concurrently.

The programming circuit 16 next determines whether the reconfigurable ultrasound array 30 is to operate in a CW mode or in a PW mode, at decision block 108. For the CW mode, the analog transmit signal 84 is provided to the low voltage switch matrix 46, at step 110. If the corresponding switch is in the “ON” state in the low voltage switch matrix 46, the analog transmit signal 84 passes through the cell T/R switch 48 on the low voltage transmit path 74. For the PW mode, the high voltage pulse transmitter 44 sends a signal on the high voltage transmit path 86, at step 112. For either the CW or PW modes, the ultrasound transducer element 34 “fires,” at step 114. The receiver “listens” for the ultrasound echo, and the process repeats steps 108 through 114 if, at decision block 116, it is determined that the imaging session has not been completed. Otherwise, control returns to the programming circuit 16, at step 118.

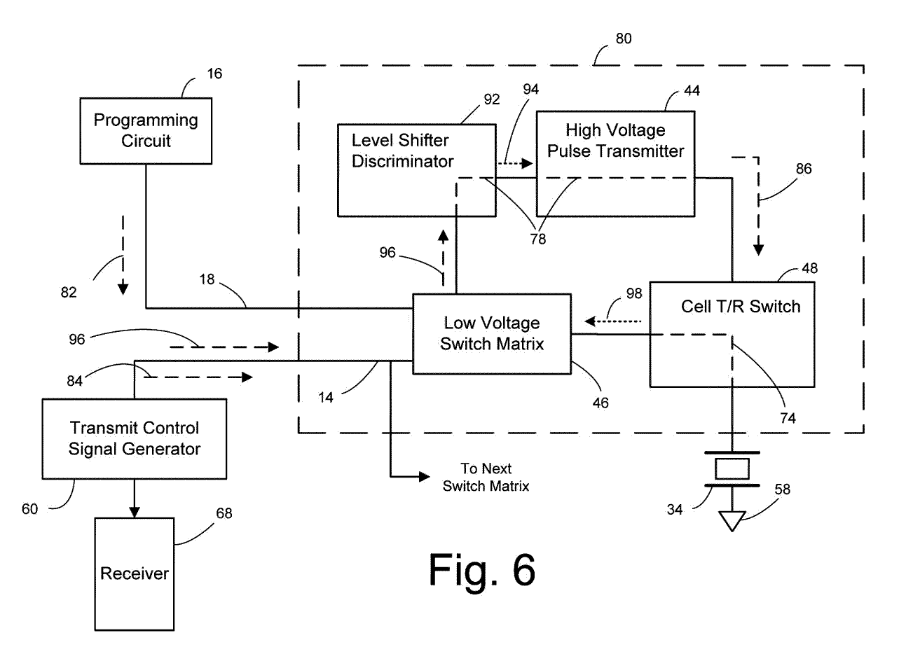

There is shown in FIG. 6 a diagrammatical illustration of an alternative exemplary embodiment of the probe system 10 in which a unit transducer cell 80 is configured for ultrasound transmission using external pulse timing. It should be understood that either the unit transducer cell 40 or the unit transducer cell 80 can be used in the reconfigurable ultrasound transducer array 30 for one or more of the unit transducer cells 40 shown in FIG. 2. A level shifter discriminator 92 in the unit transducer cell 80 functions to control the high voltage pulse transmitter 44 via a pulser control signal 94 provided in response to low voltage transmit control signal 96. The pulser control signal 94 may be transmitted to the high voltage pulse transmitter 44 via a low-voltage pulser control path 78. The transmission signal 86 is subsequently passed to the ultrasound transducer element 34, as in the unit transducer cell 40 above.

The analog transmit signals 84 generated by the transmit control signal generator 60 may be provided to the low voltage switch 46 in the unit transducer cell 90. The low voltage switch 46 can be switched to a first configuration in which the analog transmit signal 84 is passed to the cell T/R switch 48. The analog transmit signal 84 functions as described in the flow diagram 100, as in FIG. 5 above. In an alternative transmit mode of operation, the low voltage transmit control signal 96 may be provided to the low voltage switch matrix 46. Accordingly, the low voltage switch 46 can be switched to a second configuration in which the low-voltage control path 78 is provided for transmitting the pulser control signal 94 via the level shifter discriminator 92.

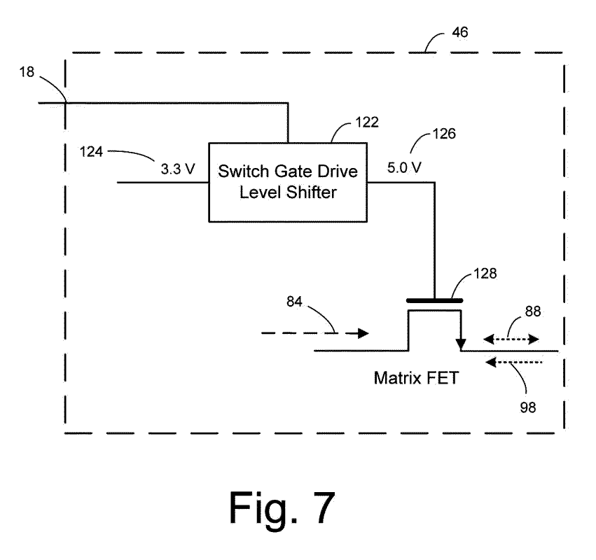

The low voltage switch matrix 46 may include a switch gate drive level shifter 122, as shown in greater detail in FIG. 7. A logic input signal 124, which can be on the order of 3.3 volts, may be provided to the switch gate drive level shifter 122 via the system channel line 18. The switch gate drive level shifter 122 may output a logic signal 126 of about 5.0 volts to a matrix FET 128 in the low voltage switch matrix 46. In turn, the matrix FET 128 may function to pass the analog transmit signals 84 to the cell T/R switch 48 via the low-voltage transmit path 74. This configuration makes it possible for the transmit control signal generator 60 to transmit continuous wave ultrasound pulses through the low voltage switch matrix 46, where the continuous wave ultrasound pulses have a greater voltage (e.g., about −5.0 volts to +5.0 volts) than a signal voltage that could otherwise be handled by the low voltage switch matrix 46.

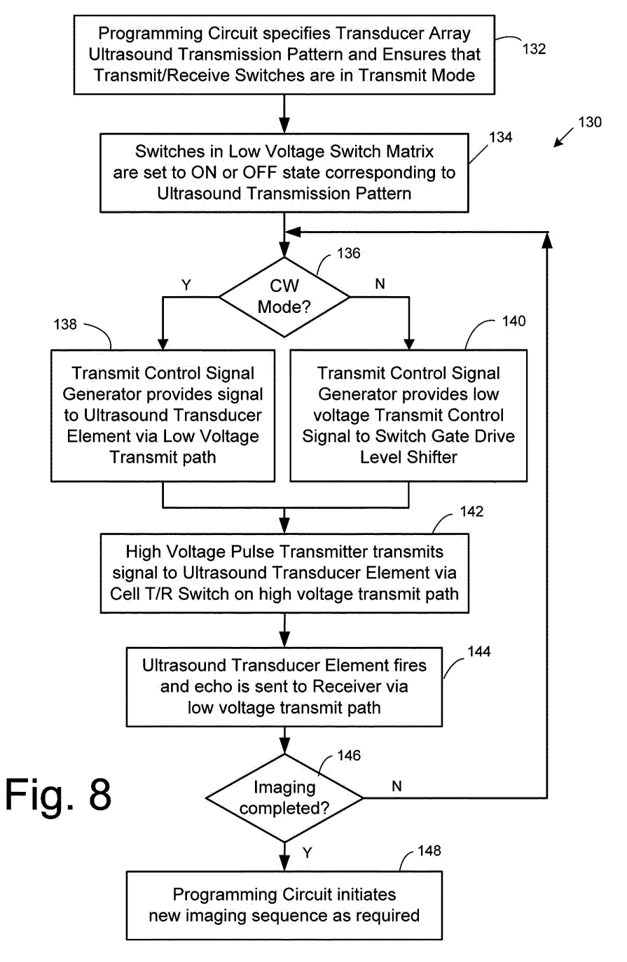

The probe system 10 may function in accordance with a flow diagram 130, shown in FIG. 8, when configured with the unit transducer cell 80 operating in the alternative transmit mode. The programming circuit 16 may verify that the cell T/R switch 48 is in a transmit mode, in step 132. Corresponding digital control signals 82 and the low voltage transmit control signal 96 are provided to the low voltage switch matrices 46 in the unit transducer cells 80 in the ultrasound transducer element array 32. Switches in each low voltage switch matrix 46 are programmed to change state to “ON” or “OFF,” or to remain unchanged in response to a “NO_CHANGE” control signal, at step 134. It should be understood that step 134 may be performed before step 132.

The programming circuit 16 next determines whether the reconfigurable ultrasound array 30 is to operate in a CW mode or in a PW mode, at decision block 136. For the CW mode, the analog transmit signal 84 is provided to the low voltage switch matrix 46, at step 138. If the corresponding switch is in the “ON” state in the low voltage switch matrix 46, the analog transmit signal 84 passes through the cell T/R switch 48 on the low voltage transmit path 74. For the PW mode, the low voltage transmit control signal 96 generated in the transmit control signal generator 60 propagates through the low voltage switch matrix 46 to the level shifter discriminator 92, at step 140. In response to receipt of the low voltage transmit control signal 96, the high voltage pulse transmitter 44 sends a signal on the high voltage transmit path 86, at step 142, and causes the ultrasound transducer element 34 to “fire,” at step 144. The receiver “listens” for the ultrasound echo, and the process repeats steps 136 through 144 if, at decision block 146, it is determined that the imaging session has not been completed. Otherwise, control returns to the programming circuit 16, at step 148.

While the invention is described with reference to an exemplary embodiment, it will be understood by those skilled in the art that various changes may be made and equivalence may be substituted for elements thereof without departing from the scope of the invention. In addition, many modifications may be made to the teachings of the invention to adapt to a particular situation without departing from the scope thereof. Therefore, it is intended that the invention not be limited to the embodiment disclosed for carrying out this invention, but that the invention includes all embodiments falling with the scope of the intended claims. Further, the use of the term “at least one” means one or more of the members of a group.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound transducer probe comprising:
an array of ultrasound transducer elements, each said ultrasound transducer element associated with a corresponding unit transducer cell for providing transmit and receive functions, each said unit transducer cell including a cell transmit/receive switch connected to a low voltage switch matrix via a low voltage transmit path; and
a plurality of microelectronic cross-point switches for switching an externally generated analog transmit signal to one or more of said low voltage transmit paths.

2. The ultrasound transducer probe of claim 1, wherein said microelectronic cross-point switches are responsive to an externally provided programming circuit signal by switching to an ON state or an OFF state, or by not switching state in response to a NO_CHANGE signal.

3. The ultrasound transducer probe of claim 1, wherein said low voltage switch matrix functions to selectively transmit said analog transmit signals to said ultrasound transducer elements.

4. The ultrasound transducer probe of claim 1, wherein said analog transmit signal comprises a voltage in the range of about −5.0 volts to about 5.0 volts.

5. The ultrasound transducer probe of claim 1, wherein said unit transducer cell comprises a high voltage pulse transmitter for providing a high-voltage pulse to said corresponding ultrasound transducer element.

6. The ultrasound transducer probe of claim 1, wherein said unit transducer cell further comprises one of a local transmit control generator and a level shifter discriminator for controlling said high voltage pulse transmitter.

7. The ultrasound transducer probe of claim 6, wherein said unit transducer cell further comprises a high voltage transmit path for passing a pulse transmission signal from said local transmit control generator to said high voltage pulse transmitter.

8. The ultrasound transducer probe of claim 6, wherein said unit transducer cell further comprises a low-voltage pulser control path for passing an externally provided pulser control signal to said high voltage pulse transmitter via said level shifter discriminator.

9. The ultrasound transducer probe of claim 1, wherein said unit transducer cell comprises a switch gate drive level shifter for outputting a logic-level signal to a FET, said FET functioning to pass said analog transmit signal to said cell transmit/receive switch.

10. The ultrasound transducer probe of claim 1, wherein said array of ultrasound transducer elements comprises a 32 by 32 array of ultrasound transducer elements.

11. The ultrasound transducer probe of claim 1, wherein one or more of said ultrasound transducer elements comprise one of a piezoelectric transducer or a capacitive micro-machined ultrasound transducer.

12. An ultrasound transducer probe system comprising:
a probe including,
an array of ultrasound transducer elements;
a plurality of unit transducer cells, each said ultrasound transducer element coupled to a corresponding unit transducer cell;
a transmit channel line for conducting analog transmit signals from an ultrasound driver to a low voltage switch matrix in each of said unit transducer cells, said low voltage switch matrix for switchably providing said analog transmit signal to a corresponding said ultrasound transducer element via a low-voltage electrical path; and
a transmit control signal generator connected to each said low voltage switch matrix by a system channel line.

13. The probe system of claim 12, further comprising a high voltage pulse transmitter, said high voltage pulse transmitter configured to receive at least one of a pulse transmission signal via a high voltage transmit path and a pulser control signal via a pulser control path.

14. The probe system of claim 12, wherein said analog transmit signal is routed to said ultrasound transducer element via a cell transmit/receive switch in each said unit transducer cell.

15. The probe system of claim 12, wherein high voltage signals are routed through a high voltage pulse transmitter to a cell transmit/receive switch in each said unit transducer cell.

16. A method for monitoring an interventional procedure inside a patient, said method comprising the steps of:
providing an ultrasound transducer probe having a reconfigurable ultrasound transducer array;
guiding said ultrasound transducer probe to a region of interest inside the patient;
providing an analog transmit signal to a transducer array through a switch matrix via a low-voltage electrical path;
providing a control signal to said reconfigurable ultrasound transducer array for electronically steering an ultrasound beam produced by said ultrasound transducer probe; and
imaging the interior of the patient via said ultrasound beam to obtain a three-dimensional, real-time image of said region of interest.

17. The method of claim 16, wherein said step of imaging comprises the step of providing a high voltage pulse signal to an ultrasound transducer element in said reconfigurable ultrasound transducer array via a high-voltage path.

18. The method of claim 16, wherein said step of imaging comprises the step of providing low voltage pulser control signal to a level shifter discriminator via a low-voltage pulser control path, said level shifter discriminator coupled to a high voltage pulse transmitter to control transmission of a high voltage pulse signal to a respective ultrasound transducer element in said reconfigurable ultrasound transducer array via a high-voltage path.

19. The method of claim 18, wherein said high voltage pulse signal comprises a voltage of from about thirty volts to about five hundred volts.

20. The method of claim 16, wherein said step of imaging the interior of the patient comprises the step of performing any one of a Trans-Esophoegeal Echocardiography procedure, an Intra-Cardiac Echocardiography procedure, and an Intra-Vascular Ultrasound procedure.
